(19) United States
(12) Patent Application Publication
(10) Pub. No.: US 2005/0191348 A1
(43) Pub. Date: Sep. 1, 2005

(54) Use of propargylamine as neuroprotective agent

(75) Inventors: Moussa B.H. Youdim, Haifa (IL); Tamar Amit, Haifa (IL); Orly Weinreb, Haifa (IL); Orit Bar-Am, Tivon (IL); Merav Yogev-Falach, Shaked (IL)

Correspondence Address: BROWDY AND NEIMARK, P.L.L.C.;624 NINTH STREET, NW, SUITE 300, WASHINGTON, DC, 20001-5303, US

(73) Assignee: Technion Research and Development Foundation Ltd., Haifa (IL)

(21) Appl. No.: 10/952,379

(22) Filed: Jan. 27, 2005

Related U.S. Application Data
(60) Provisional application No. 60/541,066, filed on Feb. 3, 2004.

(51) Int. Cl. A61K 9/20; A61K 31/135; A61K 31/13

(52) U.S. Cl. 424/464; 514/671

(57) ABSTRACT

Propargylamine and pharmaceutically acceptable salts thereof are useful for treating a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury.

### FIELD OF THE INVENTION

The present invention relates to propargylamine and use thereof in methods for treatment of neurodegenerative, demyelinating, behavior and other diseases, disorders or conditions.

Abbreviations: Aβ: amyloid-β; Aβ₁₋₄₂: amyloidogenic Aβ peptide; AD: Alzheimer's disease; APP: amyloid precursor protein; ChE: cholinesterase; ERK: extracellular signal-regulated kinase; MAO: monoamine oxidase; MAPK: mitogen-activated protein kinase; PKC: protein kinase C; sAPPα: α-soluble amyloid precursor protein.

### BACKGROUND OF THE INVENTION

Several propargylamine derivatives have been shown to selectively inhibit monoamine oxidase (MAO)-B and/or MAO-A activity and thus to be suitable for treatment of neurodegenerative diseases such as Parkinson's and Alzheimer's diseases. In addition, these compounds have been further shown to protect against neurodegeneration by preventing apoptosis.

The first compound found to selectively inhibit MAO-B was R-(−)-N-methyl-N-(prop-2-ynyl)-2-aminophenylpropane, also known as L-(−)-deprenyl, R-(−)-deprenyl, or selegiline. In addition to Parkinson's disease, other diseases and conditions for which selegiline was disclosed as being useful include: drug withdrawal (WO 92/21333, including withdrawal from psychostimulants, opiates, narcotics, and barbiturates); depression (U.S. Pat. No. 4,861,800); Alzheimer's disease and Parkinson's disease, particularly through the use of transdermal dosage forms, including ointments, creams and patches; macular degeneration (U.S. Pat. No. 5,242,950); age-dependent degeneracies, including renal function and cognitive function as evidenced by spatial learning ability (U.S. Pat. No. 5,151,449); pituitary-dependent Cushing's disease in humans and nonhumans (U.S. Pat. No. 5,192,808); immune system dysfunction in both humans (U.S. Pat. No. 5,387,615) and animals (U.S. Pat. No. 5,276,057); age-dependent weight loss in mammals (U.S. Pat. No. 5,225,446); schizophrenia (U.S. Pat. No. 5,151,419); and various neoplastic conditions including cancers, such as mammary and pituitary cancers. WO 92/17169 discloses the use of selegiline in the treatment of neuromuscular and neurodegenerative disease and in the treatment of CNS injury due to hypoxia, hypoglycemia, ischemic stroke or trauma. In addition, the biochemical effects of selegiline on neuronal cells have been extensively studied (e.g., see Tatton, 1993, and Tatton and Greenwood, 1991). U.S. Pat. No. 6,562,365 discloses the use of desmethylselegiline for selegiline-responsive diseases and conditions.

U.S. Pat. No. 5,169,868, U.S. Pat. No. 5,840,979 and U.S. Pat. No. 6,251,950 disclose aliphatic propargylamines as selective MAO-B inhibitors, neuroprotective and cellular rescue agents. The lead compound, (R)-N-(2-heptyl)methyl-propargylamine (R-2HMP), has been shown to be a potent MAO-B inhibitor and antiapoptotic agent (Durden et al., 2000).

Rasagiline, R(+)—N-propargyl-1-aminoindan, a highly potent selective irreversible monoamine oxidase (MAO)-B inhibitor, anti-Parkinson drug (Youdim et al., 2001a) has been shown to exhibit neuroprotective activity and antiapoptotic effects against a variety of insults in cell cultures and in vivo (Youdim and Weinstock, 2002a).

The mechanism underlying the neuroprotection by rasagiline has been studied in dopaminergic SH-SY5Y and PC12 cells in culture against apoptosis induced by N-methyl (R) salsolinol, the peroxynitrite donor, SIN-1(N-morpholino-sydnonimine), 6-hydroxydopamine, and serum and nerve growth factor (NGF) withdrawn (Youdim et al., 2001b; Akao et al., 1999, 2002; Maruyama et al., 2001a, 2001b, 2002).

Rasagiline and pharmaceutically acceptable salts thereof were first disclosed in U.S. Pat. No. 5,387,612, U.S. Pat. No. 5,453,446, U.S. Pat. No. 5,457,133, U.S. Pat. No. 5,576,353, U.S. Pat. No. 5,668,181, U.S. Pat. No. 5,786,390, U.S. Pat. No. 5,891,923, and U.S. Pat. No. 6,630,514 as useful for the treatment of Parkinson's disease, memory disorders, dementia of the Alzheimer type, depression, and the hyperactive syndrome. The 4-fluoro-, 5-fluoro- and 6-fluoro-N-propargyl-1-aminoindan derivatives were disclosed in U.S. Pat. No. 5,486,541 for the same purposes.

U.S. Pat. No. 5,519,061, U.S. Pat. No. 5,532,415, U.S. Pat. No. 5,599,991, U.S. Pat. No. 5,744,500, U.S. Pat. No. 6,277,886, U.S. Pat. No. 6,316,504, US 133, U.S. Pat. No. 5,576,353, U.S. Pat. No. 5,668,181, U.S. Pat. No. 5,786,390, U.S. Pat. No. 5,891,923, and U.S. Pat. No. 6,630,514 disclose R(+)—N-propargyl-1-aminoindan and pharmaceutically acceptable salts thereof as useful for treatment of additional indications, namely, an affective illness, a neurological hypoxia or anoxia, neurodegenerative diseases, a neurotoxic injury, stroke, brain ischemia, a head trauma injury, a spinal trauma injury, schizophrenia, an attention deficit disorder, multiple sclerosis, and withdrawal symptoms.

U.S. Pat. No. 6,251,938 describes N-propargyl-phenylethylamine compounds, and U.S. Pat. No. 6,303,650, U.S. Pat. No. 6,462,222 and U.S. Pat. No. 6,538,025 describe N-propargyl-1-aminoindan and N-propargyl-1-aminotetralin compounds, said to be useful for treatment of depression, attention deficit disorder, attention deficit and hyperactivity disorder, Tourette's syndrome, Alzheimer's disease and other dementia such as senile dementia, dementia of the Parkinson's type, vascular dementia and Lewy body dementia.

Previous work by the inventors has suggested that rasagiline and related propargylamine derivatives suppress apoptotic death cascade initiating in the mitochondria; they prevented pre-apoptotic decline in mitochondrial membrane potential (ΔΨm) due to permeability transition and the activation of the following apoptotic processes: activation of caspase 3, nuclear translocation of glyceraldehyde-3-phosphate dehydrogenase, and nucleosomal DNA fragmentation (Youdim and Weinstock, 2002b). In controlled monotherapy and as an adjunct to L-dopa, rasagiline has shown anti-Parkinson activity. Rasagiline's Phase III clinical trials in Parkinson's disease, have been successfully completed in 2003 by Teva Pharmaceutical Industries Ltd. (Petach Tikvah, Israel).

Recently, Prof. Youdim and his colleagues have synthesized two analogs from rasagiline that contain a carbamate moiety in an attempt to combine the MAO inhibitory and neuroprotective properties of rasagiline with the cholinesterase (ChE)-inhibiting activity of rivastigmine, a drug with proven efficacy in Alzheimer's disease (AD) patients; they are TV3326 [(N-propargyl-(3R)aminoindan-5yl)-ethyl methyl carbamate], which possesses both ChE and MAO-A and B inhibitory activities, and its S-isomer, TV3279, an inhibitor of ChE but not of MAO (Weinstock, 1999; Grossberg and Desai, 2001). Similar to rasagiline, these compounds possess neuroprotective properties against a variety of insults, which are independent of the ChE and MAO inhibitory activities, but may derive from some intrinsic pharmacological activity of the propargylamine moiety (Youdim and Weinstock, 2002a). In addition, TV3326 and TV3279 stimulate the release of the neurotrophic/neuroprotective nonamyloidogenic-soluble amyloid precursor protein (sAPPβ) via activation of the protein kinase C (PKC) and mitogen-activated protein kinase (MAPK) pathways (Yogev-Falach, 2002). Thus, these drugs may affect the formation of potentially amyloidogenic derivatives and could be of clinical importance for treatment of AD.

Propargylamine was reported many years ago to be a mechanism-based inhibitor of the copper-containing bovine plasma amine oxidase (BPAO), though the potency was modest. U.S. Pat. No. 6,395,780 discloses propargylamine as a weak glycine-cleavage system inhibitor.

### SUMMARY OF THE INVENTION

In one aspect, the present invention provides a method for treating a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury, which comprises administering to an individual in need an amount of propargylamine or a pharmaceutically acceptable salt thereof effective to treat said disease, disorder or condition in the individual.

In yet another aspect, the present invention relates to an article of manufacture comprising packaging material and a pharmaceutical composition contained within the packaging material, said pharmaceutical composition comprising propargylamine or a pharmaceutically acceptable salt thereof, and said packaging material includes a label that indicates that said agent is therapeutically effective for treating a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury.

### BRIEF DESCRIPTION OF THE FIGURES

FIGS. 1A-1B show the neuroprotective effect of rasagiline against serum deprivation. PC12 cells (1A) and SH-SY5Y neuroblastoma cels (1B) were incubated under serum deprivation and were untreated or treated with rasagiline (0.1, 1, 10 μM) for 24 hours. Cell death was detected by ELISA and the values are expressed as a percentage of the control. The results are the mean±SEM (n=3). *P<0.05 vs. control, untreated cells.

FIGS. 2A-2CB show the effect of rasagiline on protein kinase C (PKC) activation. (2A) PC12 cells were untreated or treated with various concentrations of rasagiline (0.1, 1 and 10 μM) fir 1 hour. (2B) PC12 cells were pre-incubated with vehicle alone, or with GF 109203×, a potent and selective inhibitor of PKC (2.5 μM), and then incubated without or with rasagiline (1 μM) or PMA (100 nM) for 1 h. Phosphorylation of PKC was analyzed in cell lysates by Western blotting and detected with p-PKC (pan) antibody. The loading of the lanes was normalized to levels of β-actin. Results are representative of 3 independent experiments. (2C) Immunoreactivity of the p-PKC (pan) and PKCα and PKCε isoenzymes present in the membrane fractions of PC12 cells untreated (control) or treated with rasagiline (1 μM) for 1 hour. The results are the mean±SEM (n=3−5). **P<0.01; ***P<0.001 vs. the respective control cells.

FIGS. 3A-3D show the neuroprotective effects of propargylamine against serum deprivation. PC12 cells (3A) and SH-SY5Y neuroblastoma cells (3B) were incubated under serum deprivation and were untreated or treated with propargylamine (0.1, 1, 10 μM) for 24 hours. Cell death was detected by ELISA and the values are expressed as a percentage of the control. The results are the mean±SEM (n=3). *p<0.05 vs. control, untreated cells. (3C) Morphological characteristics visualized by phase-contrast microscope of PC12 cells maintained with full serum medium (a), or serum-free medium in the absence (b) or presence of propargylamine (1 μM) (c) or (10 μM) (d). The morphology was visualized by phase-contrast microscope. The results are representative photographic. Similar results were repeated 3 times. (3D) Effect of propargylamine on caspase-3 cleavage in SH-SY5Y neuroblastoma cells incubated 3 days in serum free medium, followed by 48 h incubation with or without propargylamine (1,10 μM).

FIGS. 4A-4B show the effect of propargylamine on PKC phosphorylation. (3A) PC12 cells were untreated or treated with various concentrations of propargylamine (0.1, 1 and 10 μM) for 1 hour. (3B) PC12 cells were pre-incubated for 30 min with vehicle alone or with GF 109203X (2.5 μM), and then incubated without or with propargylamine (1 μM) for 1 h. Phosphorylation of PKC was analyzed in cell lysates by Western blotting using p-PKC (pan) antibody. The loading of the lanes was normalized to levels of β-actin. Results are representative of 3 independent experiments.

FIGS. 5A-5B show the effect of rasagiline on Bcl-2 family gene expression: Bcl-xL, Bcl-W, Bad and Bax. PC12 cells were maintained in full-serum (control), or in serum-free (SF) media in the absence or presence of various concentrations of rasagiline (0.001, 0.1, 1 and 10 μM) for 3 h (5A) or 24 h (5B), and gene expression was measured by quantitative real-time RT-PC. The amount of each product was normalized to the housekeeping gene 18S-rRNA and expressed as fold stimulation of control, arbitrarily set as 1. Results are representative of 3 independent experiments, performed in duplicates and exhibited similar results. Data are expressed as the mean±SD. t-test (#P<0.05 vs. control; *P<0.05 vs. SF).

FIGS. 6A-6B show the effect of propargylamine on Bad (6A) and Bax (6B) gene level. PC12 cells were maintained in full-serum (control), or in serum-free (SF) media in the absence or presence of various concentrations of propargylamine (0.1, 1, 10 μM) for 24 h, and gene expression was measured by quantitative real-time RT-PCR. The amount of each product was normalized to the housekeeping gene 18S-rRNA and expressed as fold stimulation of control, arbitrarily set as 1. Results are representative of 3 independent experiments, performed in duplicates and exhibited similar results. Data are expressed as the mean±SD. t-test (#P<0.05 vs. control; *P<0.05 vs. SF).

FIGS. 7A-7C show the neuroprotective effect of rasagiline against Aβ-induced toxicity. PC12 cells were pretreated without or with rasagiline (1 and 10 μM) and then incubated for 24 h without or with Aβ₁₋₄₂ (10 μM). (7A) Cell viability was estimated by directly counting cell numbers by using the Trypan blue dye exclusion method. The values are expressed as a percentage of viable cells counted in the control. (7B) Cell death was detected by ELISA. The values are expressed as percentage of the control. The results are the mean±S E (n=3). *P<0.05; **P<0.001 vs.control, untreated cells; # P<0.05 vs. Aβ₁₋₄₂-induced cell death. (7C) depicts morphological characteristics visualized by phase-contrast microscopy of PC12 cells treated for 24 h (a) without rasagiline (control), or with (b) rasagiline (1 μM), (c) rasagiline (10 μM), (d) Aβ₁₋₄₂ (10 μM), (e) rasagiline (1 μM) plus Aβ₁₋₄₂ (10 μM), and (f) rasagiline (10 μM) plus Aβ₁₋₄₂ (10 μM). The morphology was visualized by phase-contrast microscope. The results are representative photographs of experiments repeated three times.

FIG. 8 shows the effect of rasagiline on sAPPα release in SH-SY5Y neuroblastoma cells. SH-SY5Y neuroblastoma cells were incubated for 3 h without or with increasing concentrations of rasagiline (0.1, 1, and 10 μM), and sAPPα was measured in the media as described in Materials and Methods. Densitometric analysis of Western blots is expressed as a percentage of basal sAPPα release. Results are shown as the mean±SE values of (n=3−5) independent experiments. *P<0.05;**P<0.001 vs. control.

FIG. 9 shows the effect of rasagiline on sAPPα release in PC12 cells. PC12 cells were incubated for 3 h without or with increasing concentrations of rasagiline (0.1, 1, and 10 μM), and sAPPα was measured in the media as described in Materials and Methods. Densitometric analysis of Western blots is expressed as a percentage of basal sAPPα release. Results are shown as the mean±SE values of (n=3−5) independent experiments. *P<0.05;**P<0.001 vs. control.

FIGS. 10A-10B show the effect of rasagiline on MAPK activation. (10A) PC12 cells were treated for 0.5 h with increasing concentrations of rasagiline (0.1, 1 and 10 μM). Quantification of the phospho-MAPK blots were normalized using the total MAPK blots. (10B) PC12 cells were preincubated for 15 min with vehicle alone or with PD98059 (30 μM) or GF109203X (2.5 μM) and then incubated without or with rasagiline (10 μM) for 0.5 h. Aliquots of cell lysates were then subjected to immunoblot analysis and probed with anti-phospho-MAPK (top blots) and anti-MAPK (bottom blots). Data are mean±SEM values of (n=3) independent experiments. **P<0.001 vs. control.

FIGS. 11A-11B show the effect of propargylamine on sAPPα release. SH-SY5Y neuroblastoma cells (11A) or PC12 cells (11B) were incubated for 3 h without or with increasing concentrations of propargylamine (0.1, 1 and 10 μM), and sAPPα was measured in the media as described in Materials and Methods. Densitometric analysis of Western blots was expressed as a percentage of basal sAPPα release. Results are shown as the mean±SEM of (n=3−5) independent experiments. *P<0.05; **P<0.001 vs. control.

FIG. 12 shows the effect of propargylamine on MAPK activation. PC12 cells were treated for 0.5 h with increasing concentrations of propargylamine (0.1, 1 and 10 μM). Aliquots of cell lysates were then subjected to immunoblot analysis and probed with anti-phospho-MAPK (top blots) and anti-MAP kinase (bottom blots). Quantification of the phospho-MAPK were normalized using the total MAPK blots. Data are mean±SE values of (n=3) independent experiments. **P<0.001 vs. control.

FIGS. 13A-13B show that propargylamine inhibits both MAO-A (13A) and MAO-B (13B) activity in a dose-dependent manner, but is relatively selective for MAO-B.

### DETAILED DESCRIPTION OF THE INVENTION

In previous preliminary studies, we speculated that the propargylamine moiety of the anti-Alzheimer drugs (N-propargyl-(3R)aminoindan-5yl)-ethyl methyl carbamate (TV3326) and its S-isomer (TV3279) might be pharmacologically responsible for MAPK-dependent APP processing (Yogev-Falach et al., 2002).

According to the present invention, we have determined the effects of rasagiline, its S-optical isomer TVP1022 and their metabolite, aminoindan (devoid of propargylamine moiety), and propargylamine itself on the regulation of APP processing and the signaling pathways that are involved, using cultured human SH-SY5Y neuroblastoma and rat phaeochromocytoma PC12 cells. It is shown herein that both rasagiline and propargylamine induce the release of the non-amyloidogenic α-secretase form of soluble APP by MAPK- and PKC-dependent mechanisms in cell-cultures. Moreover, rasagiline and its derivatives regulate PKC-related mechanisms and APP processing in vivo.

One of the principal molecular components of the apoptosis programme in neurons are the proteins of the Bcl-2 family that may either support cell survival (Bcl-2, Bcl-xL, Bcl-w, Mcl-1, A1/Bfl-1) or promote cell death (Bax, Bak, Bcl-Xs, Bad, Bid, Bik, Hrk, Bok).

It appears that members of the Bcl-2 family interact among each other to form a dynamic equilibrium between homo- and heterodimers. Because members of those opposing factions can associate and seemingly titrate one another's function, their relative abundance in a particular cell type may determine its threshold for apoptosis. The competitive action of the pro- and anti-survival Bcl-2 family proteins regulates the activation of the proteases (caspases) that dismantle the cell.

According to the present invention, we further examined whether Bcl-2 family proteins mediate the pro-survival effect of rasagiline and propargylamine using serum-starved rat pheochromocytoma PC12 cell line, as a model of neuronal apoptosis. Moreover, the involvement of the PKC cascade in rasagiline- and propargylamine-mediated neuroprotection processes was determined.

We have thus determined according to the present invention that propargylamine itself exhibits neuroprotective and anti-apoptotic activities and can, therefore, be used for all known and future uses of rasagiline and similar drugs containing the propargylamine moiety.

The present invention provides pharmaceutical compositions comprising a pharmaceutically acceptable carrier and propargylamine or a physiologically acceptable salt thereof.

The use of any physiologically acceptable salt of propargylamine is encompassed by the present invention such as the hydrochloride, hydrobromide, sulfate, mesylate, esylate, tosylate, sulfonate, phosphate, or carboxylate salt. In more preferred embodiments, propargylamine hydrochloride and propargylamine mesylate are used according to the invention.

The pharmaceutical composition provided by the present invention may be in solid, semisolid or liquid form and may further include pharmaceutically acceptable fillers, carriers or diluents, and other inert ingredients and excipients. The composition can be administered by any suitable route but will preferably be administered orally as tablets or capsules. The dosage will depend of the state of the patient and severity of the disease and will be determined as deemed appropriate by the practitioner.

Due to its neuroprotective and antiapoptotic activity, propargylamine can be used for treatment, including prevention, of a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury, which comprises administering to an individual in need an amount of propargylamine or a pharmaceutically acceptable salt thereof effective to treat said disease, disorder or condition in the individual.

In one embodiment, the invention relates to a method for treatment of a neurodegenerative disease such as, but not limited to, Parkinson's disease, Huntington's disease and amyotrophic lateral sclerosis. In these cases, the treatment may involve the prevention of further neurodegeneration and the further progress of the disease.

In another embodiment, the invention relates to a method for treatment of a dementia that can be Alzheimer's dementia or a non-Alzheimer's dementia selected from the group consisting of Lewy body dementia, vascular dementia and a dementia caused by any other disease, disorder or condition such as Parkinson's disease, Huntington's disease, Creutzfeldt-Jakob disease, head trauma or HIV infection.

In a further embodiment, the invention relates to a method for treatment of an affective or mood disorder such as, but not limited to, depression, a dysthymic disorder, a bipolar disorder, a cyclothymic disorder, schizophrenia or a schizophrenia-related disorder selected from the group consisting of brief psychotic disorder, a schizophreniform disorder, a schizoaffective disorder and delusional disorder. In preferred embodiments, propargylamine is used for the reatment of depression or schizophrenia.

In still another embodiment, the invention relates to a method for treatment of drug use and dependence such as, but not limited to, alcoholism, opiate dependence, cocaine dependence, amphetamine dependence, hallucinogen dependence, or phencyclidine use and withdrawal symptoms related thereto.

In still a further embodiment, the invention relates to a method for treatment of a memory loss disorder such as amnesia or memory loss associated with Alzheimer's type dementia, with a non-Alzheimer's type dementia, or with a disease or disorder selected from the group consisting of Parkinson's disease, Huntington's disease, Creutzfeldt-Jakob disease, head trauma, HIV infection, hypothyroidism and vitamin B12 deficiency.

In yet another embodiment, the invention relates to a method for treatment of an acute neurological traumatic disorder or neurotrauma such as head trauma injury or spinal cord trauma injury.

In yet a further embodiment, the invention relates to a method for treatment of multiple sclerosis, a demyelinating disease.

In a further embodiment, the invention relates to a method for treatment of a seizure disorder such as epilepsy.

In another embodiment, the invention relates to a method for treatment of a cerebrovascular disorder such as brain ischemia or stroke.

In a further embodiment, the invention relates to a method for treatment of a behavior disorder of neurological origin such as a hyperactive syndrome or an attention deficit disorder.

In a yet a further embodiment, the invention relates to a method for treatment of a neurotoxic injury, for example, caused by a neurotoxin, defined herein as any substance which possesses the ability to damage or destroy nerve tissue such as a nerve gas used in chemical warfare or the toxin delivery system of poisonous snakes, fish or animals.

The invention will now be illustrated by the following non-limiting examples.

### EXAMPLES

Materials and Methods

(i) Materials. Rasagiline [N-propargyl-(1R)-aminoindan] and propargylamine were kindly donated by Teva Pharmaceutical Industries Ltd. (Petach Tikva, Israel). Electrophoresis reagents were obtained from InVitrogen Corporation (Carlsbad, Calif., USA). The PKC inhibitor GF109203X and the MAPK inhibitor PD98059 were obtained from Calbiochem (La Jolla, Calif., USA) dissolved (1×100) in dimethyl sulfoxide and stored at −20° C. Tissue culture reagents were from Beth-Haemek (Israel).

Antibodies against p-PKC (pan) were from Cell Signaling Technology (Beverly, Mass., USA) and those against PKCα and PKCε were purchased from BD Biosciences Pharmingen (Heidelberg, Germany). Monoclonal antibodies 22C11 (to APP N terminus) were purchased from Roche Molecular Biochemicals (Mannheim, Germany), and 6E10 (which recognize an epitope within residues 1-17 of Aβ domain of APP) was from Senetek (St. Louis, Mo.). Anti-phospho-MAPK and anti-MAPK antibodies were purchased from Cell Signaling Technology (MA). Aβ₁₋₄₂ was purchased from Bachem (Bubendorf, Switzerland).

β-Actin antibody, Tri Reagent™ RNA Isolation Reagent and all other chemicals were purchased from Sigma Chemical Co. (St Louis, Mo., USA). RT reaction mix, random hexanucleotides, dNTP, RNasin inhibitor and M-MLV reverse transcriptase were purchased from Promega (Madison, Wis., USA). The LightCycler—DNA Master SYBR Green I ready-to use reaction mix for PCR Kit for DNA amplification and detection containing FastStart (Hot Start) Taq DNA polymerase was purchased from Roche Diagnostica GmBH (Mannheim, Germany).

(ii) Cell culture, viability measurements and experimental treatments. Two different neuronal cell lines were used: the rat PC12 and the human neuroblastoma SH-SY5Y cells. Both cells have been widely used for neurological and neurochemical studies, including studies involving APP regulation because they constitutively express significant levels of APP and release considerable amounts of nontoxic, nonamyloidogenic soluble APPα.

SH-SY5Y neuroblastoma cells were plated in 100-mm culture dishes and cultured in Dulbecco's modified Eagle's medium (DMEM; 4500 mg/l glucose), containing 10% fetal calf serum (FCS) and a mixture of 1% penicillin/streptomycin/nystatin.

PC12 cells, originated from rat pheochromocytoma, were grown to confluence in T75 flasks containing DMEM (1000 mg/l glucose) and supplemented with 5% FCS, 10% horse serum, and a mixture of 1% of penicillin/streptomycin/nystatin.

Cell cultures were incubated at 37° C. in a humid 5% CO₂-95% air environment. For cell viability assays, cells were treated with various drugs in serum free media containing 0.1% BSA.

For the experiments, cells were seeded (2×10⁶) onto 100-mm plates (for SH-SY5Y cells) or T75 flasks (for PC12 cells) in culture medium supplemented with full serum for 48 h. The cells were replaced with fresh medium containing 0.5% FCS for an additional 24 h (subconfluent culture), and then the experiments were performed in fresh medium containing 0.5% FCS for the indicated time periods at 37° C. After incubation with various drugs, the conditioned media were collected, centrifuged at 3500 g (10 min, 4° C.) to remove cell debris, and the cleared supernatants were concentrated 10-fold by lyophilization.

(iii) Measurement of cell death. PC12 cells were detached by vigorous washing, centrifuged at 200 g for 5 min, and resuspended in DMEM with 2% FCS. The cells were plated in a microtiter plate (96 wells) at a density of 1.5-2×10⁴ cells/well and were allowed to attach for 24 h before treatment. Trypan blue exclusion or a cell death detection ELISA kit (Roche Molecular Biochemicals, Indianapolis, Ind., USA) were used to detect apoptosis after Aβ₁₋₄₂ treatment. The ELISA was performed according to the manufacturer's protocol, based on a quantitative sandwich ELISA, using antibodies directed against DNA and histones to detect mono- and oligonucleosomes in the cytoplasm of cells undergoing apoptosis. The absorption was determined in a Tecan Sunrise Elisa-Reader (Hombrechtikon, Switzerland) at λ=405/490 nm after automatic subtraction of background readings. Results are expressed as percentage of the control values. In addition, neuronal cell injury was evaluated by a colorimetric assay for mitochondrial function by using 3-(4,5-dimethylthiazol-2-yl)-2,5-diphenyl-tetrazolium bromide (MTT-test). The absorption was determined in a Tecan Sunrise Elisa-Reader at λ=570/650 nm after automatic subtraction of background values. Neuronal cell injury was also evaluated by morphological characteristics, visualized by phase-contrast microscopy.

(iv) Immunoblot Analyses. For Western blot analyses, attached cells were washed once in cold PBS and then lysed in 1× nonreducing Laemmli buffer followed by boiling for 3-5 min. Protein content was determined using the Bradford method. Equal amounts of protein were separated by SDS-polyacrylamide gel electrophoresis and blotted onto polyvinylidene difluoride (PVDF) membranes (Millipore). Membranes were treated with blocking buffer (5% dry milk in PBS or 5% dry milk, 0.05% Tween 20 (Sigma) in TBS). Primary antibodies were diluted in PBS or in TBS containing 5% BSA, 0.05% Tween 20 and incubated with membranes for 20 h at 4° C., followed by incubation (1 h at room temperature) in dilutions of horseradish peroxidase (HRP)-conjugated secondary antibodies in the same buffers. Following antibody incubations, membranes were washed in 0.5% Tween 20 in PBS or TBS. Detection was achieved using ECL™ Western Blotting Detection Reagent (Amersham, Pharmacia, Little Chalfort Buckinghamshire, UK). Quantification of results was accomplished by measuring the optical density of the labeled bands from the autoradiograms by scanning laser densitometry using the computerized imaging program Bio-ID (Imager, Bio1D software; Vilber Lourmat, Marne La Vallee, France). The values were normalized to β-actin intensity levels.

(v) PKC translocation. Cells were cultured to confluence and treated for 2 h with either no drug (control), phorbol 12-myristate-13-acetate (PMA) (positive control) or rasagiline. After washing, the cells were harvested, collected by centrifugation (5 min, 1500 rpm), and homogenized by sonication (12 s, 22μ) in homogenization buffer (20 mM Tris-HCl, pH 7.4, 0.32 M sucrose, 2 mM EDTA, 50 mM β-mercaptoethanol) containing a protease inhibitor cocktail (Sigma Aldrich, Sweden). Cytosolic and particulate fractions were separated by ultracentrifugation (30 min, 100,000 g, 4° C.). For the particulate fraction, proteins were extracted by a 45 min incubation on ice in homogenization buffer containing 0.5% Triton X-100. Samples were then sonicated for 1 min and equivalent amounts of protein (15 μg) separated by PAGE followed by blotting onto nitrocellulose paper. Western blotting was performed essentially as described above using antibodies against p-PKC (pan), PKCα and PKCε.

(vi) Total RNA extraction. PC12 cells were cultured in DMEM containing 5% FCS and 10% horse serum until confluence. At the day of the experiment, the medium was replaced with fresh medium without serum and rasagiline or propargylamine at various concentrations were administered for 24 h. At the end of incubation, the medium was removed and isolation of total RNA was performed, using Tri Reagent™ Isolation reagent. Total RNA was treated with DNase-RNase free (Roche Diagnostics, Mannheim, Germany) for 30 min at 37° C. and subsequently extracted by a round of phenol:chlorophorm:isoamylalcohol (25:24:1), followed by one of chloroform. After precipitation with NaOAc (0.3 M) and ethyl alcohol, the RNA pellet was washed with 80% ethyl alcohol (12,000×g) for 10 min and resuspended in 50-100 μl of diethylpyrocarbonate (DEPC)-treated water and incubated for 5-10 min at 56° C. to facilitate resuspension.

(vi) Reverse transcription (RT) and quantitative real-time RT-PCR. In order to confirm the results obtained with the customized cDNA expression micro-array, 2 μg of total RNA were denatured and reverse transcribed using random hexanucleotides (0.5 μg/μl). Secondary structures of the template and primer (total volume of 16 μl) were opened by incubation for 5 min at 70° C. and immediately cooled on ice. Reaction mix (9 μl) containing reaction buffer, dNTP (0.5 mm each), RNAasin™ RNAase inhibitor (Promega) (25U) and M-MLV reverse transcriptase (200U) was added and samples were incubated at 39° C. for 1 h. For every RNA preparation a negative control was run in parallel consisting of a direct amplification of the RNA sample, omitting the RT step. The samples were transferred to 92° C. for 10 min, in order to deactivate the enzyme, and then cooled to 4° C.

Quantitative real-time PCR, using LightCycler and FastStart DNA Master SYBR Green I ready-to use PCR mix, was performed according to the manufacture's protocol (Roche Diagnostics, Mannheim, Germany). cDNA (40 ng) was amplified in 20 μl total volume. The sequences of the primers and the size of the products are described in Table 1. The results are analyzed on the provided program of the LightCycler. The relative expression level of a given mRNA was assessed by normalizing to the housekeeping gene 18S-rRNA and compared with control values.

(vii) Determination of sAPP. Protein concentrations of cell lysates were assayed using Bradford reagent (Sigma Chemical). Equal amounts of volumes of conditioned medium, standardized to lysate protein, were subjected to sodium dodecyl sulfate (SDS)-nitrocellulose membranes, using either the MAbs 22C11 or 6E10 in 50 mM Tris-HCl, pH 7.4, 150 mM NaCl, containing 0.05% Tween 20 and 1% BSA. Immunoreactivity was detected using anti-mouse IgG-conjugated with HRP and an enhanced chemiluminescence method (Amersham, Pharmacia Biotech, UK). Blots were developed for different times to span a linear range of response, and the relative intensity of immunoreactive bands on the exposed films was quantified by computer-assisted densitometry program (Quantity One, Bio-Rad, Hercules, Calif., USA).

(viii) Extracellular signal-regulated kinase (ERK) activity. The kinase activity of the ERKs was measured using the MAPK assay kit (Cell Signaling Technology) according to the manufacturer's protocol. In brief, for kinase activity assays, PC12 cells were grown in 6-well plates (5×10⁵ cells/well). Eighteen to twenty-four hours before experiments, the medium was replaced with DMEM with 0.5% FCS and treated as described in the figures. Experimental treatments were performed in 1 ml medium containing 0.5% FCS, with the vehicle or test drugs and/or inhibitors at 37° C., as described in the figures. After treatment, reactions were stopped by placing cells on ice and aspirating the medium. Cells were harvested in 50 mM Tris-HCl, pH 8.0, 150 mM NaCl, 5 mM EDTA, 1 mM DTT, 1% Triton X-100, and protease- and phosphatase-inhibitor cocktails. Protein concentration was determined by the Bradford method. As described previously (Yogev-Falach et al., 2002), each cell lysate, containing 30 μg protein, was separated on 4-12% SDS-polyacrylamide electrophoresis gels, immunoblotted, and identified using phospho-p44/42 MAPK (Thr202/Tyr204) antibody or p44/42 MAPK antibody. Data are representative of three to six independent experiments.

TABLE 1
Sequences of primers for quantitative real-time PCR

| | | | Size of |
|---|---|---|---|
| | Oligonucleotide sequence (5′-3′) | Oligonucleotide sequence (5′-3′) | product |
| mRNA | Forward | Reverse | (bp) |
| Bad | GCT TAG CCC TTT TCG AGG AC | GAT CCC ACC AGG ACT GGA T | 200 |
| | (SEQ ID_NO:1) | (SEQ ID_NO:2) | |
| Bax | TGC AGA GGA TGA TTG CTG AC | GGA GGA AGT CCA GTG TCC AG | 207 |
| | (SEQ ID_NO:3) | (SEQ ID_NO:4) | |
| Bcl-W | GCT GAG GCA GAA GGG TTA T | TGG AAA AGT TCG TCG GAA AC | 201 |
| | (SEQ ID_NO:5) | (SEQ ID_NO:6) | |
| Bcl-xL | GGT GAG TCG GAT TGC AAG TT | GAG CCC AGC AGA ACT ACA CC | 198 |
| | (SEQ ID_NO:7) | (SEQ ID_NO:8) | |
| BDNF | GAC TCT GGA GAG CGT GAA T | CCA CTC GCT AAT ACT GTC AC | 325 |
| | (SEQ ID_NO:9) | (SEQ ID_NO:10) | |
| PKC-α | TGA ACC CTC AGT GGA ATG AGT CCT | ATG GCT GCT TCC TGT CTT CTG AAG | 326 |
| | (SEQ ID_NO:11) | (SEQ ID_NO:12) | |
| PKC-γ | TTC TTC AAG CAG CCA ACC TT | TGT AGC TGT GCA GAC GGA AC | 202 |
| | (SEQ ID_NO:13) | (SEQ ID_NO:14) | |
| PKC-δ | AAA TAG AGA GCG CAG CCT GA | TAA ACA AGG CCG AAT GTT CC | 206 |
| | (SEQ ID_NO:15) | (SEQ ID_NO:16) | |
| PKC-ε | CCC CTT GTG ACC AGG AAC TA | GCC TTT GCC TAA CAC CTT GA | 203 |
| | (SEQ ID_NO:17) | (SEQ ID_NO:18) | |
| 18S-rRNA | GTAACCCGTTGAACCCCATT | CCATCCAATCGGTAGTAGCG | 170 |
| | (SEQ ID_NO:19) | (SEQ ID_NO:20) | |

All templates were initially denatured for 3 min at 95° C. Amplification was done for 45 cycles. In order to receive melting temperatures of the products, melting curve analysis was done by continuous acquisition from 65° C. to 95° C. with temperature transition rate of 0.1° C./sec.

### EXAMPLE 1

### Rasagiline Prevents Loss of Viability in Serum-Deprived Cells

In order to test cell viability, PC12 cells and SH-SY5Y neuroblastoma cels were incubated under serum deprivation and were untreated or treated with rasagiline (0.1. 1 or 10 μM) for 24 hours.

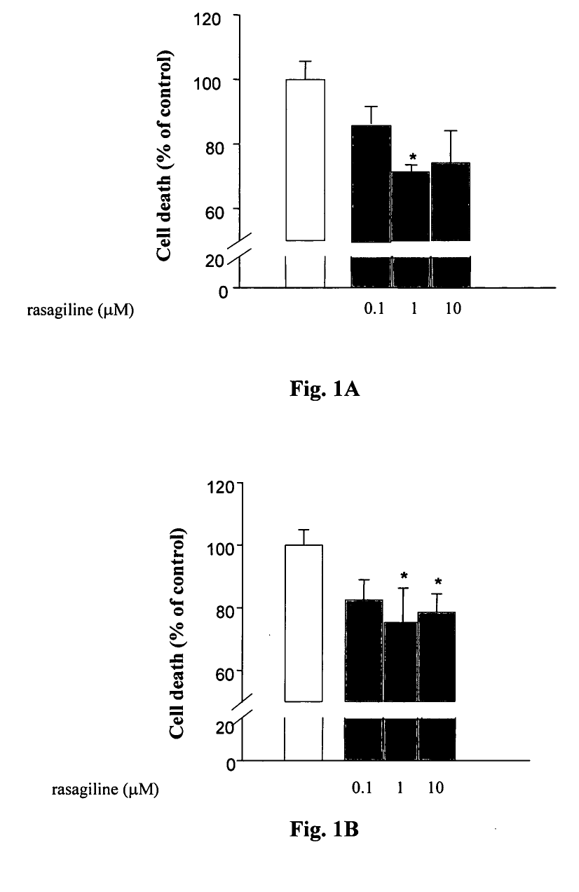

As shown in FIGS. 1A-1B, cell viability of untreated PC12 and SH-SY5Y neuroblastoma cells was markedly reduced by 24 h serum withdrawal. Rasagiline significantly reduced cell death induced by serum deprivation in PC12 cells (FIG. 1A) and in SH-SY5Y neuroblastoma cells (FIG. 1B), as assessed by an apoptotic cell death detection ELISA. Similar results were obtained by the MTT reduction analysis (data not shown). Significant survival effects of rasagiline were observed at 1 and 10 μM.

Consistent with its impact on apoptosis, rasagiline decreased serum free-induced cleavage and activation of caspase-3 and the suppressive effect of rasagiline was reversed by the PKC inhibitor, GF109203X (data not shown). Similarly, rasagiline decreased serum free-induced levels of the important regulator of the cell death machinery, BAD, and this effect was blocked by GF109203X. These data suggest the involvement of PKC-dependent pathway in rasagiline-stimulated cell viability.

### EXAMPLE 2

### Rasagiline Affects PKC Activation

Since activation of PKC has been shown previously to modulate cell viability, resulting in protection of neuronal cells, it was of interest to investigate the effects of rasagiline on PKC activation. In one experiment, PC12 cells were untreated or treated with various concentrations of rasagiline (0.1, 1 and 10 μM) for 1 hour. In other experiments, PC12 cells were pre-incubated with vehicle alone, or with GF 109203X, a potent and specific broad spectrum PKC inhibitor (2.5 μM), and then incubated without or with rasagiline (1 μM) or PMA (100 nM) for 1 h. Phosphorylation of PKC was analyzed in cell lysates by Western blotting and detected with p-PKC (pan) antibody.

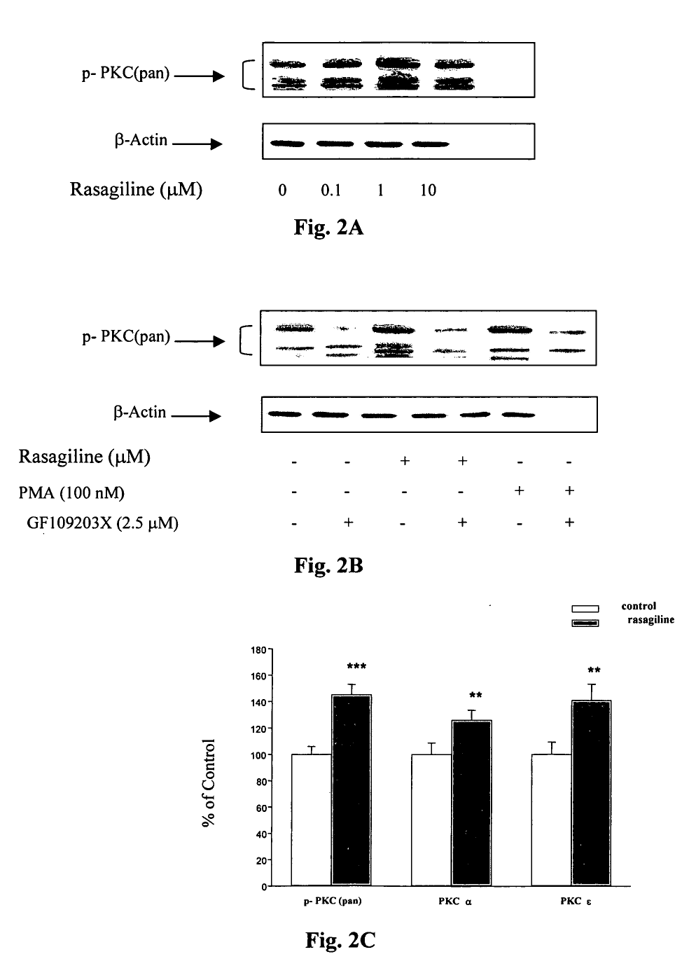

The results, depicted in FIGS. 2A-2B, demonstrate that short-term treatment of PC12 cells with rasagiline (1 h) dose-dependently induced PKC phosphorylation (2A), and pre-treatment with GF109203X (2.5 μM), a specific broad spectrum PKC inhibitor, significantly reduced rasagiline-induced PKC phosphorylation (2B).

The effects of rasagiline treatment on the subcellular redistribution of p-PKC (pan), PKCα and PKCε, were studied next to establish further the activation of PKC and to obtain data on the isoforms that may be involved in the effect of rasagiline. PKC translocation to the membrane fraction upon activation and membrane localization is often used as a marker for PKC activation.

In preliminary studies, using PMA as a positive control, we found that exposure of PC12 cells to PMA (100 nM) for 2 h markedly induced the translocation of p-PKC (pan), PKCα and PKCε (214%, 157% and 212%, respectively). These data are consistent with early studies, showing that PMA time-dependently induced membrane translocation of PKC isoforms in PC12 cells.

Stimulation with rasagiline (1 μM) lead also to p-PKC (pan) as well as PKCα and PKCε translocation to the membrane fractions (FIG. 2C), suggesting that these isoforms are activated by rasagiline.

### EXAMPLE 3

### Propargylamine Prevents Loss of Viability in Serum-Deprived Cells

In order to test cell viability, PC12 cells and SH-SY5Y neuroblastoma cels were incubated under serum deprivation and were untreated or treated with propargylamine (0.1. 1 or 10 μM) for 24 hours. Apoptotic cell death was detected by ELISA and the values are expressed as a percentage of the control.

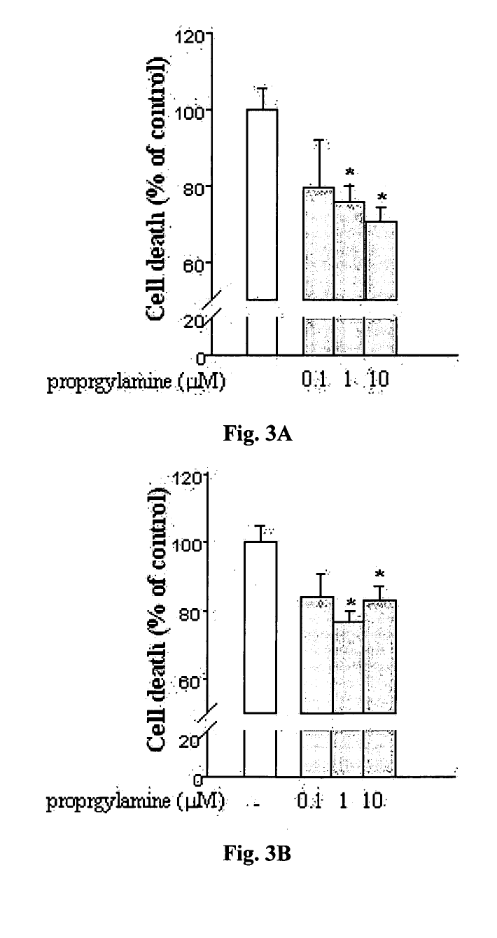

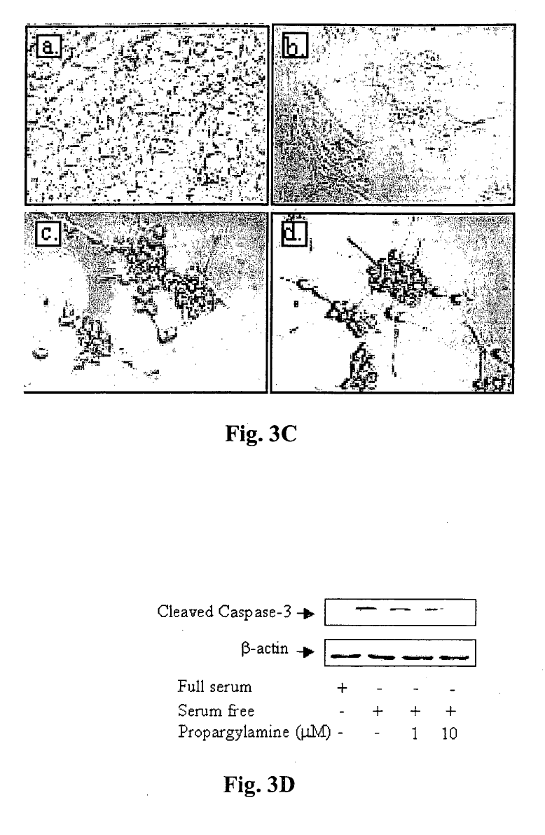

As shown in FIGS. 3A-3B, propargylamine (1 and 10 μM) significantly reduced cell death induced by serum deprivation in both PC12 (3A) and SH-SY5Y neuroblastoma (3B) cells. Consistent with this result, propargylamine also decreased activated caspase-3 (3D). Activation of caspase 3 has become almost synonymous with cell death and caspase 3 cleavage is an essential part of a neuroprotective effect. FIG. 3C shows morphological characteristics visualized by phase-contrast microscopy of PC12 cells maintained with full serum medium (a), or serum-free medium in the absence (b) or presence of propargylamine (1 μM) (c) or (10 μM) (d).

### EXAMPLE 4

### Propargylamine Affects PKC Activation

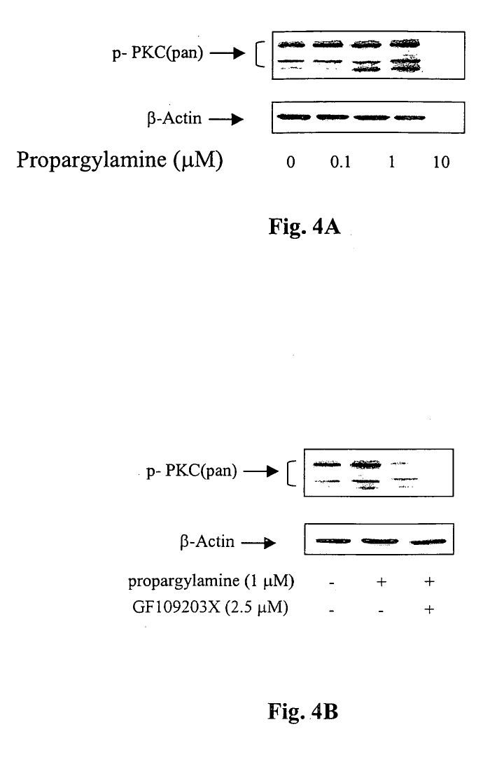

The effect of propargylamine on PKC phosphorylation/activation was investigated as described in Example 2 above. The results, depicted in FIGS. 4A-4B, show that treatment of PC12 cells with increasing concentrations of propargylamine resulted in a significant, dose-dependent increase in PKC phosphorylation (4A), and pre-treatment with GF109203X (2.5 μM) blocked the effect of propargylamine on PKC phosphorylation and significantly reduced the propargylamine-induced PKC phosphorylation (4B).

### EXAMPLE 5

### Rasagiline Modifies Bcl-2 Family mRNA Levels

One of the principal molecular components of the apoptosis programmed in neurons are the proteins of the Bcl-2 family that may either support cell survival (Bcl-2, Bcl-xL, Bcl-w, Mcl-1, A1/Bfl-1) or promote cell death (Bax, Bak, Bcl-Xs, Bad, Bid, Bik, Hrk, Bok). The competitive action of the pro- and anti-survival Bcl-2 family proteins regulates the activation of the proteases (caspases) that dismantle the cell.

In this experiment, we examined whether Bcl-2 family proteins mediate the pro-survival effect of rasagiline, using serum-starved rat pheochromocytoma PC12 cell line as a model of neuronal apoptosis. PC12 cells were maintained in full-serum (control), or in serum-free (SF) media in the absence or presence of various concentrations of rasagiline (0.001, 0.1, 1 and 10 μM) for 3 h or 24 h, and gene expression was measured by quantitative real-time RT-PC. The real-time RT-PCR consists of RNA samples isolated from said PC-12 cells. Each gene expression was normalized to the housekeeping gene, 18S-rRNA, since this transcript was reported to be less susceptible to the influence by external factors.

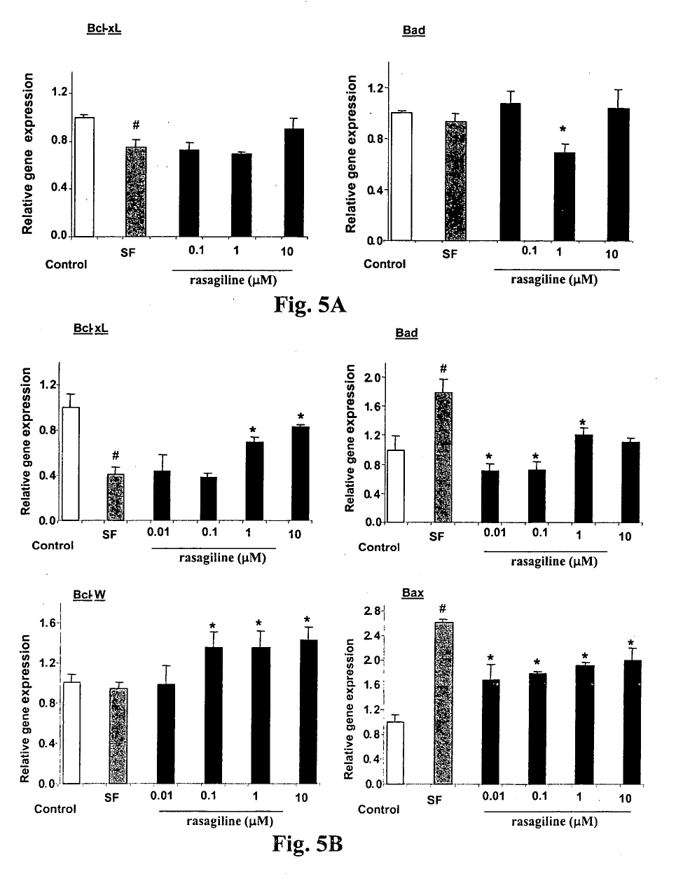

As shown in FIGS. 5A-5B, real-time RT-PCR analysis revealed up-regulation of pro-apoptotic gene expression and down-regulation of anti-apoptotic gene expression after treatment with rasagiline, in a time- and concentration-dependent manner. The anti-apoptotic Bcl-xL mRNA expression began to increase after 3 h of the treatment with 10 μM rasagiline (FIG. 5A), and the increase continued further to about 2-fold at 24 h, compared to the down-regulated Bcl-xL levels observed in serum-free culture (40% of full serum culture) (FIG. 5B). A delayed and less-marked induction in Bcl-xL expression was observed with 1 μM rasagiline at 24 h of incubation, partially restored to the expression levels observed in full serum culture. Significant enhancement was also seen in mRNA expression of another anti-apoptotic member, Bcl-W, after 24 h treatment with 0.1, 1 and 10 μM rasagiline (135% of serum-free culture). While the expression levels of the pro-apoptotic Bax in serum-free culture were markedly increased (260% vs. full serum culture), rasagiline significantly decreased mRNA levels of Bax. Similarly, the proapoptotic gene, Bad was substantially reduced (1.35-fold vs. serum free treatment) by 1 μM rasagiline, evident as early as 3 h after treatment (FIG. 5A). At 24 h, 0.01 and 0.1 μM rasagiline reduced Bad expression in a concentration-dependent profile (˜2.5-fold vs. serum free culture) and thus restoring Bad gene level to that of full serum level (FIG. 5B). The genes, Bcl-W and Bax, were not significantly modulated after 3 h of incubation (data not shown).

### EXAMPLE 6

### Effect of Propargylamine on Bax and Bad mRNA Levels

The effect of propargylamine on Bad and Bax mRNA levels was examined as described in Example 5 above.

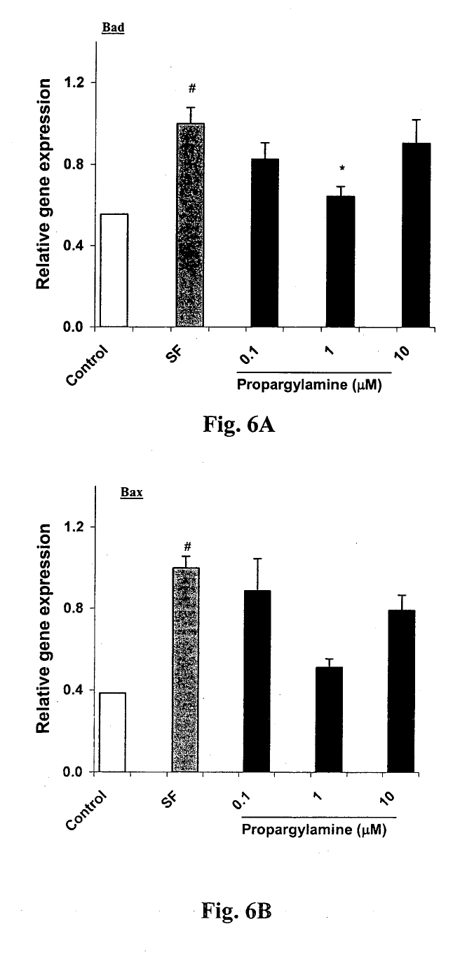

As shown in FIGS. 6A-6B, real-time RT-PCR analysis demonstrate that propargylamine (1 μM) restored both Bax and Bad gene expressions to that of full serum level.

### EXAMPLE 7

### Neuroprotective Effect of Rasagiline Against Aβ₁₋₄₂ Toxicity

Because Aβ peptides have been shown to be highly toxic to a variety of primary neurons and neuronal cell lines, we characterized the response of rat PC12 neuronal cells to Aβ₁₋₄₂ peptide using the MTT method, as described previously (Yogev-Falach et al., 2002). Treatment of PC12 cells with Aβ₁₋₄₂ (1-50 μM) for 24 h caused a dose-dependent loss of cell viability being maximal at 10 μM Aβ₁₋₄₂ (data not shown).

Rasagiline has been previously shown to exhibit neuroprotective and anti-apoptotic activity against several neurotoxins (Youdim and Weinstock, 2002a). To evaluate whether rasagiline could also prevent Aβ-induced toxicity, we preincubated PC12 cells for 1 h in the absence or in the presence of rasagiline (1 and 10 μM), then incubated the cells for 24 h without or with aggregated Aβ₁₋₄₂ (10 μM), and assessed cell death.

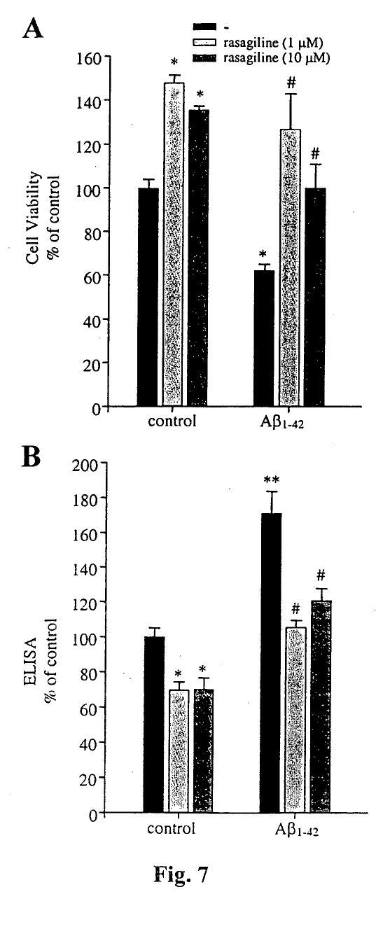

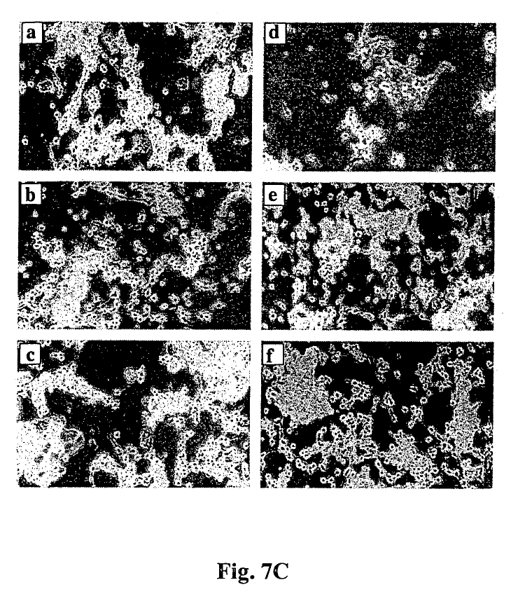

The results are shown in FIGS. 7A-7C. In FIG. 7A, cell viability was estimated by direct counting cell numbers by using the Trypan blue dye exclusion method, and in FIG. 7B, cell death was assessed by ELISA. In both figures, the values are expressed as a percentage of viable cells counted in the control. FIG. 7C depicts morphological characteristics visualized by phase-contrast microscopy of PC12 cells treated for 24 h (a) without rasagiline (control), or with (b) rasagiline (1 μM), (c) rasagiline (10 μM), (d) Aβ₁₋₄₂ (10 μM), (e) rasagiline (1 μM) plus Aβ₁₋₄₂ (10 μM), and (f) rasagiline (10 μM) plus Aβ₁₋₄₂ (10 μM). The results in FIGS. 7A-7C show that Aβ₁₋₄₂ markedly reduced cell viability (66% of control), whereas rasagiline significantly protected (P<0.05) PC12 cells against Aβ₁₋₄₂ neurotoxicity. Rasagiline alone (1 and 10 μM) also induced a significant improvement in PC12 cell viability, which is presumably due to its effect on cell survival after serum reduction and withdrawal (Tatton et al., 2002).

### EXAMPLE 8

### Effect of Rasagiline and its Derivatives on sAPPα Processing

We have recently reported that (N-propargyl-(3R)aminoindan5-yl)-ethyl methyl carbamate (TV3326), which was synthesized from rasagiline for the treatment of Alzheimer's disease, stimulates the release of sAPPα (Yogev-Falach et al., 2002). It was of interest to determine whether rasagiline and other propargylamine-containing drugs also regulate APP processing. For this purpose, SH-SY5Y neuroblastoma cells were incubated for 3 h without or with 1 μM of various drugs (TV3326 and its S-isomer TV3279, rasagiline and its S-isomer TVP1022, selegiline, aminoindan), and sAPPα was measured in the media as described in Materials and Methods. The results in Table 2 demonstrate that all the tested compounds, with the exception of aminoindan, significantly induced sAPPα release.

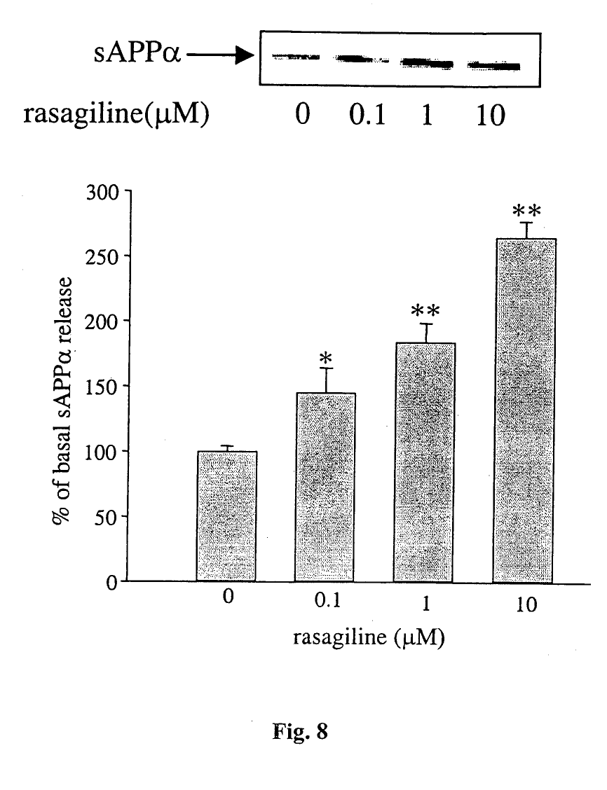

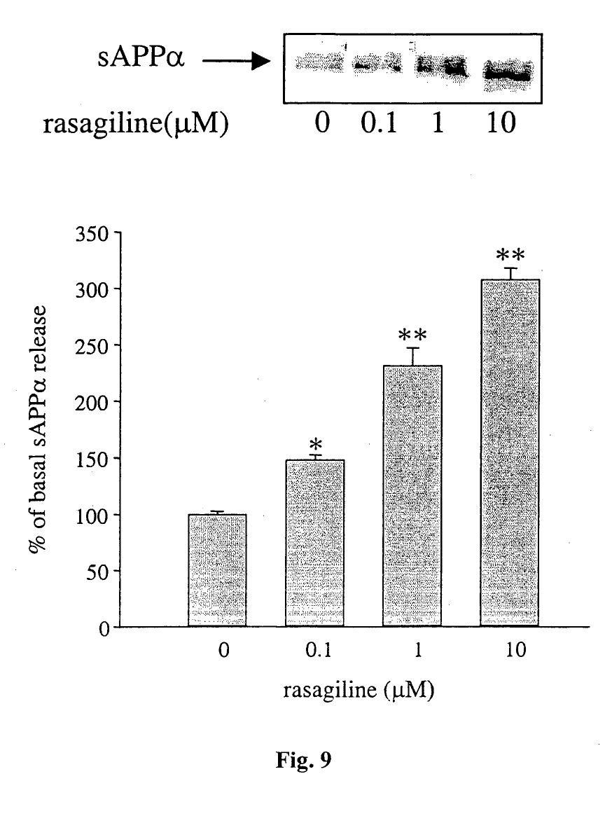

We further characterized, in detail, the effect of rasagiline on APP processing and the signaling pathways that are involved. Thus, SH-SY5Y neuroblastoma cells were incubated for 3 h without or with increasing concentrations of rasagiline (0.1, 1 and 10 μM), and sAPPα was measured in the media as described in Materials and Methods. The results in FIG. 8 show that treatment of SH-SY5Y neuroblastoma cells for 3 h with increasing concentrations of rasagiline resulted in a significant dose-dependent increase in sAPPα released into the medium, compared with the level in control, untreated cells. The maximal effect was obtained at a concentration of 10 μM, which resulted in a ˜3.0-fold increase in sAPPα secretion over the basal levels. PC12 cells treated for 3 h with various doses of rasagiline also showed a concentration-dependent release of sAPPα (FIG. 9), the maximal stimulation effect being observed again at the concentration of 10 μM.

### EXAMPLE 9

### Activation of MAPK by Rasagiline

To study the effect of rasagiline on MAPK activation, PC12 cells were treated for 0.5 h with increasing concentrations of rasagiline (0.1, 1 and 10 μM), and quantification of the phospho-MAPK blots were normalized using the total MAPK blots, or PC12 cells were preincubated for 15 min with vehicle alone, or with PD98059 (30 μM) or GF109203X (2.5 μM) and then incubated without or with rasagiline (10 μM) for 0.5 h. Aliquots of cell lysates were then subjected to immunoblot analysis and probed with anti-phospho-MAPK (top blots) and anti-MAPK (bottom blots).

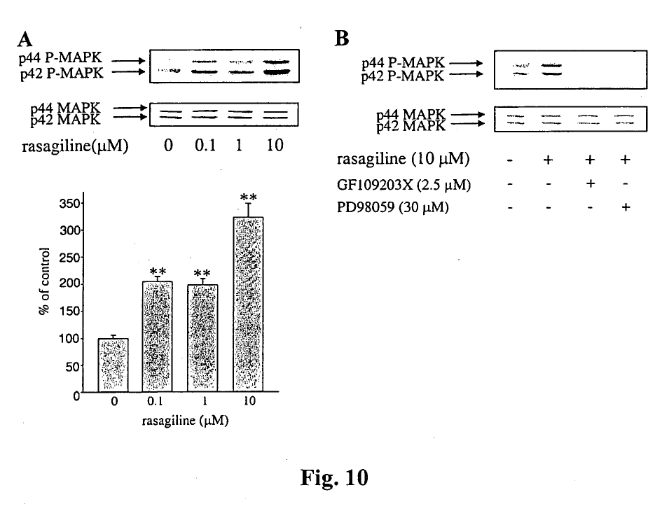

As shown in FIG. 10A, rasagiline dose-dependently increased the immunoreactivity of the phosphorylated MAPK in PC12 cells, but had no effect on total levels of MAPK proteins. Activation occurred with doses as low as 0.1 μM of rasagiline and maximal activation at 10 μM. To determine the inhibitory potential of a noncompetitive inhibitor of MEK phosphorylation and activation, the effect of PD98059 on rasagiline-induced phosphorylation of MAPK was examined. As shown in FIG. 10B, pretreatment with PD98059 (30 μM) blocked the rasagiline-induced increase of MAPK phosphorylation.

We also examined the role of PKC signaling pathway in rasagiline-stimulated MAPK activation, using the specific PKC-inhibitor GF109203X (2.5 μM). As shown in FIG. 10B, preincubation with GF109203X abolished the effect of rasagiline on MAPK activation, suggesting the involvement of PKC in rasagiline-induced MAPK activation.

### EXAMPLE 10

### Effects of Propargylamine on sAPPα Processing and MAPK Activation

To determine its role on APP processing, the effects of propargylamine on sAPPα release and MAPK activation were assessed.

For the sAPPα release, SH-SY5Y neuroblastoma cells or PC12 cells were incubated for 3 h without or with increasing concentrations of propargylamine (0.1, 1 and 10 μM), and sAPPα was measured in the media as described in Materials and Methods.

For the effect of propargylamine on MAPK activation, PC12 cells were treated for 0.5 h with increasing concentrations of propargylamine (0.1, 1 and 10 μM). Aliquots of cell lysates were then subjected to immunoblot analysis and probed with anti-phospho-MAPK and anti-MAP kinase. Quantification of the phospho-MAPK were normalized using the total MAPK blots.

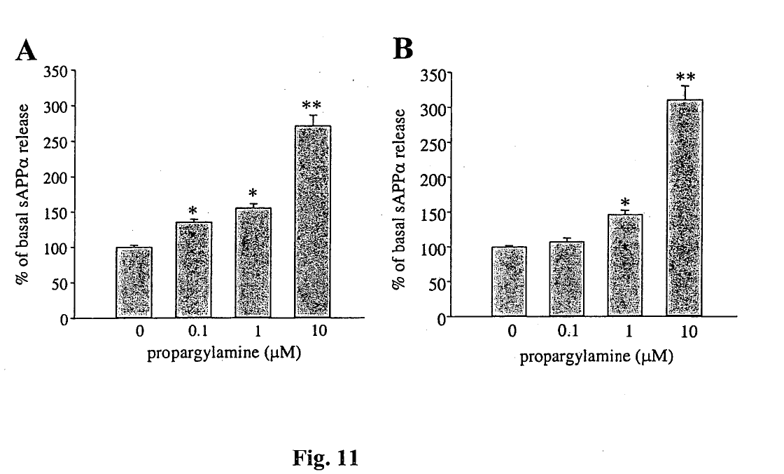

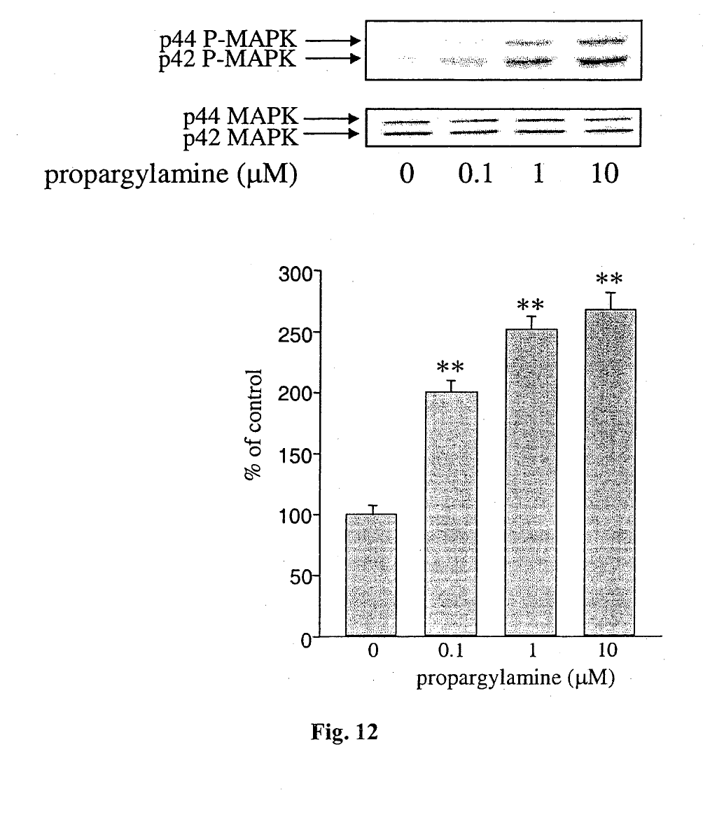

FIGS. 11A-11B show that treatment of SH-SY5Y neuroblastoma cells and PC12 cells, respectively, with increasing concentrations of propargylamine (0.1, 1 and 10 μM), resulted in a significant, dose-dependent increase in sAPPα into the medium, compared with the level in control, untreated cells. The maximal effect was obtained at a concentration of 10 μM. Furthermore, as shown in FIG. 12, based on immunoblot analysis with anti-phospho-p44/p42 MAPK, propargylamine, dose-dependently, induced MAPK phosphorylation in PC12 cells. These findings indicate that the effects of propargylamine on both sAPPα release and MAPK activation are similar to those of rasagiline, TV3326, and TV3279. However, the metabolite of rasagiline, aminoindan (TVP136), that lacks the propargyl group, did not show such effects on either sAPPα release (Table 2) or MAPK activation (data not shown).

TABLE 2
Effect of propargyl-containing drugs and aminoindan on sAPPα release^{a}

| Treatment | sAPPα secretion |
|---|---|
| (1 μM) | (fold/basal) |
| TV3326 | 2.86 ± 0.35^{b} |
| TV3279 | 2.76 ± 0.36^{b} |
| Rasagiline | 2.97 ± 0.15^{b} |
| TVP1022 | 2.43 ± 0.27^{c} |
| Selegiline | 2.03 ± 0.21^{c} |
| Aminoindan | 1.23 ± 0.32 |

^{a}SH-SY5Y neuroblastoma cells were incubated for 3 h without or with 1 μM of various drugs, and sAPPα was measured in the conditioned media. Densitometric analysis of Western blots, expressed as fold/basal sAPPα release and as mean ± SE of three independent experiments.
^{b}P < vs. control.
^{c}P < 0.05.

### EXAMPLE 11

### In Vitro Inhibitory Activity of Propargylamine Against Rat Brain Monoamine Oxidase (MAO)

Monoamine oxidase (MAO), an enzyme that plays a crucial role in the metabolic degradation of biogenic amines, exists in two functional forms: MAO-A and MAO-B. The two isoenzymes have different substrate and inhibitor specificities. Selective MAO-B inhibitors have great potential for treating Parkinson's disease.

To determine the activity of MAO-A and MAO-B, the test compound is added to a suitable dilution of the enzyme preparation from rat brain (70 μg protein for MAO-B and 150 μg MAO-A assay) in 0.05 M phosphate buffer (pH 7.4). Tthe mixture is incubated together with 0.05 M deprenyl/selegiline, a specific inhibitor of MAO-B (for determination of MAO-A) or with 0.05 M clorgylin, a specific inhibitor of MAO-A (for determination of MAO-B). Incubation is carried for 1 h at 37° C. before addition of ¹⁴C-5-hydroxytryptamine binoxalate (100 M), for determination of MAO-A, or ¹⁴C-phenylethylamine (100 M), for determination of MAO-B, and incubation is continued for 30 min or 20 min, respectively. The reaction is stopped with 2 M ice-cold citric acid, and the metabolites are extracted and determined by liquid-scintillation counting in cpm units.

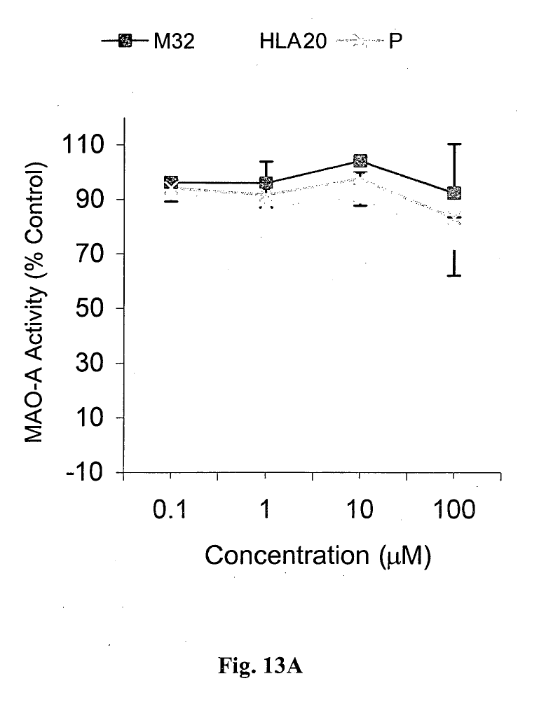

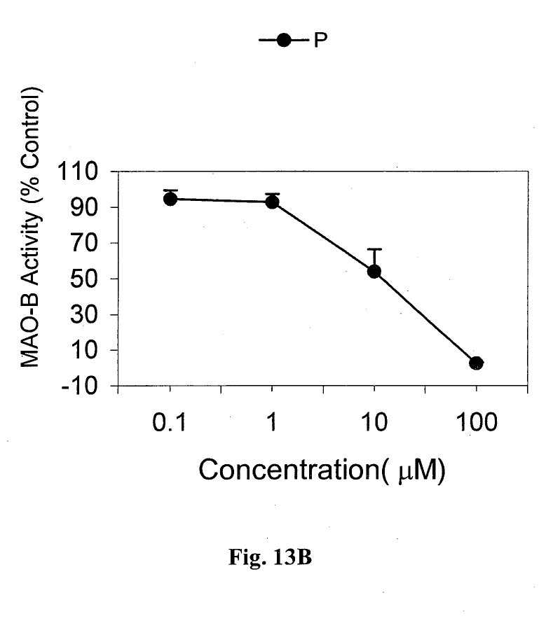

Thus, in one experiment, the in vitro inhibitory activity of 5-(4-propargylpiperazin-1-ylmethyl)-8-hydroxy-quinoline (HLA20) and the 8-hydroxyquinoline derivative designated M32 (both iron chelators disclosed in the International Application PCT/IL03/00932), and propargylamine (P) (0.1, 1, 10, 100 μM) were tested against rat brain MAO-A. The test compounds were added to buffer containing 0.05 μM deprenyl and were incubated with the tissue homogenate for 60 min at 37° C. before addition of ¹⁴C-5-hydroxytryptamine. The results are shown in FIG. 13A. MAO-A activity in presence of the test compound was expressed as a percentage of that in control samples. Mean values shown±SEM.

In another experiment, the in vitro inhibitory activity of propargylamine (P) against rat brain MAO-B was tested. Propargylamine (0.1, 1, 10, 100 μM) was added to buffer containing 0.05 μM clorgylin and was incubated with the tissue homogenate for 60 minutes at 37° C. before addition of ¹⁴C-5-phenylethylamine. The results are shown in FIG. 13B. MAO-BA activity in presence of the test compound was expressed as a percentage of that in control samples. Mean values shown±SEM.

As shown in FIGS. 13A-B, propargylamine inhibited both MAO-A and MAO-B activity in a dose-dependent manner, with the IC₅₀ values about 66 μM and 10 μM, respectively, indicating that it is preferentially selective for MAO-B inhibition.

### DISCUSSION

We show here, for the first time, that the anti-Parkinson-propargylamine-containing MAO-B inhibitor drug, rasagiline, has neuroprotective properties against Aβ-induced cytotoxicity. Furthermore, our results demonstrate that rasagiline induces sAPPα release and ERK activation, and it is suggested that the mechanism of action is attributed to the propargylamine moiety in the molecule.

Rasagiline has been shown to have potent neuroprotective-antiapoptotic activity in PC-12 cells deprived of serum and NGF and against endogenous neurotoxin N-methyl-R-salsolinol (Akao et al., 2002a; Maruyama et al., 2001b, 2001c); 6-hydroxydopamine (Maruyama et al., 2000a); SIN-1, a peroxynitrite donor (Maruyama et al., 2002); glutamate toxicity in rat hippocampal primary neurons (Finberg et al., 1999); and in several in vivo models, including N-methyl-4-phenyl-1,2,3,6-tetrahydropyridine (MPTP), global ischaemia, and closed head injury (Speiser et al., 1999; Huang et al., 1999). The antiapoptotic action of the drug is dependent on synthesis of new proteins, which are prevented by the transcriptional and translational inhibitors cycloheximide and actinomycin, respectively, in partially neuronally differentiated PC12 cells (Maruyama et al., 2000b). The mechanism of the antiapoptotic effect of rasagiline against these neurotoxins and the identity of the antiapoptotic proteins synthesized have been shown to be related to the ability of rasagiline to enhance the expression of the antiapoptotic Bcl-2 and Bcl-xL in neuroblastoma SH-SY5Y cells (Akao et al., 2002b). Thus, as has been previously demonstrated, rasagiline protects not only cell viability but also cell function (Youdim and Weinstock, 2002).

However, it is unlikely that its neuroprotective effect is related to MAO-B inhibition, because PC12 cells contain MAO type A rather than type B (Youdim, 1991) and the range of concentrations used in the experiments herein is not sufficient to inhibit MAO-A (Youdim et al., 2001a, 2001b). Moreover, the S-isomer of rasagiline, TVP1022, which is not an inhibitor of MAO-A or of -B (Youdim et al., 2001a, 2001b), also protected PC12 cells from Aβ-evoked apoptosis, suggesting that the mode of action is independent of MAO inhibition. These results are consistent with previous reports providing clear evidence that the neuroprotection by rasagiline and its derivatives does not depend on inhibition of MAO-B (Youdim et al., 2001b) but rather is associated with some intrinsic pharmacological action of the propargyl moiety in these compounds acting on the mitochondria cell survival proteins.

We have studied herein whether rasagiline, a noncholinergic drug, can also regulate APP processing and investigated the signaling pathways that are involved in its action. In our investigation we have observed that short treatment (3 h) with rasagiline can affect APP metabolism by stimulating sAPPα release in SH-SY5Y neuroblastoma and PC12 cells. Increased sAPPα secretion was detected by the mAb 22C11, as well as by the mAb 6E10, which recognizes α-secretase-cleaved APP, and the increase was dose-dependent.

Among the mechanisms that regulate proteolytic APP processing, activation of PKC and PKC-coupled receptors was shown to increase the generation of sAPP derived by α-secretase cleavage. The data presented here demonstrate that sAPPβ release, induced by rasagiline, was modulated by inhibitors of PKC and the ERK MAPK signaling pathway. Moreover, in results complementary to the inhibitor studies, we found that rasagiline dose-dependently increased the immunoreactivity of the phosphorylated MAPK in PC12 cells. Western blot analysis, using a phosphospecific MAPK antibody, revealed a concentration-dependent increase in MAPK phosphorylation in cells stimulated with rasagiline. The MEK inhibitors, PD98059, antagonized MAPK activation, indicating that MEK phosphorylates MAPK in the presence of rasagiline. The specific PKC inhibitor, GF109203X, which indicated the dependence on PKC signaling pathway activity, also effectively attenuated activation of MAPK.

We have demonstrated in the experiments herein that rasagiline, its S-isomer TVP1022, and propargylamine (that lacks cholinergic activity) stimulated sAPPβ release and MAPK phosphorylation, and we suggest that these effects do not appear to result from ChE-inhibitory activities. Furthermore, by comparing the actions of rasagiline with those of its S-isomer, TVP1022, which is at least 1000-fold weaker as an inhibitor of MAO (Youdim et al., 2001b), we have been able to demonstrate that MAO-B inhibition is not a prerequisite for either the neuroprotection effect against Aβ toxicity or the sAPPβ-induced release. Indeed, the structure-activity relationship among rasagiline-related compounds, suggests the crucial role of the propargyl moiety on these molecules for processing of APP, because propargylamine itself induced the secretion of the nonamyloidogenic β-secretase form of the sAPP into the conditioned media of SH-SY5Y neuroblastoma and PC12 cells, and significantly increased MAPK phosphorylation with similar potency to that of rasagiline and its derivatives.

We have further demonstrated in the experiments herein that the neuroprotective effect of rasagiline appears to involve activation of the pro-survival PKC pathway and regulation of Bcl-2 family anti-apoptotic related genes.

Given that the neuroprotective action of rasagiline was blocked by inhibition of PKC activity, clearly supports a role of PKC activation in the neuroprotective mechanisms that are activated by the drug to counteract apoptotic signals in neuronal cells. In results complementary to the inhibitor studies, we found that rasagiline can activate PKCα and PKCε in serum-deprived PC12 cells, being isoforms essentially involved in cell survival pathways. Furthermore, real time PCR analyses revealed that exposure of serum-deprived PC12 cells to rasagiline markedly increased PKCα and PKCε gene expression.

More recent work from the inventors has shown that rasagiline up-regulated phospho(p)-PKC level and the expression of α and ε isoenzymes in mice hippocampus. Indeed, studies investigating the role of PKC family in the regulation of cell death have suggested that activation of these PKC isoforms can prevent apoptosis. For instance, PKCα was shown to phosphorylate Bcl-2 in a site that increases its anti-apoptotic function, over expression of PKCε results in increased expression of Bcl-2 and suppression of PKCα triggers apoptosis through down-regulation of Bcl-XL.

In addition, MAPK/ERK cascades, which have been shown to inhibit apoptosis in a number of systems, can be activated by PKC. Thus, PKCα phosphorylates and activates raf-1, an upstream kinase in the MAPK/ERK pathway, PKCε and δ regulate ERK-1 and -2 activation; and pharmacological inhibition of MAPK/ERK signaling blocks phorbol ester-induced protection of neuronal cells against glutamate toxicity. In accordance, the MAPK/ERK cascade was recently found to be regulated by rasagiline.

By contrast, PKCγ mRNA levels were found increased in serum-deprived PC12 cells and rasagiline significantly reversed this induction. In supporting of these results, previous studies indicated that PKCγ was increased in ischemia and decreased by the immunosuppressant cerebroprotective agent, FK506. However, it is unlikely that its neuroprotective effect is related to MAO-B inhibition, because PC12 cells contain MAO type A rather than type B and the range of concentrations used in this study is not sufficient to inhibit MAO-A. Moreover, the S-isomer of rasagiline, TVP1022, which is not an inhibitor of MAO-A or of -B, also protected serum deprived PC12 cells from apoptosis, suggesting that the mode of action is independent of MAO inhibition. These results are consistent with previous reports providing clear evidence that the neuroprotection by rasagiline and its derivatives does not depend on inhibition of MAO-B but rather is associated with some intrinsic pharmacological action of the propargyl moiety in these compounds acting on the mitochondria cell survival proteins.

Further, the inventors demonstrated in the experiments herein the crucial role of the propargyl moiety in rasagiline, and showed according to the invention that propargylamine itself significantly augmented the viability of serum-deprived cells and induced PKC activation with similar potency to that of rasagiline. Consistent with these data, the inventors' recent study on structure—activity relationship among rasagiline-related compounds also revealed the importance of the propargyl moiety. Thus, similar to rasagiline and its derivatives, propargylamine induced the secretion of sAPPα and increased MAPK phosphorylation.

Real-time PCR analyses of the Bcl2-related protein family gene expression provided evidence for modulation of a number of mRNAs by rasagiline, including a decrease in mRNA of the proapoptotic Bax and Bad and an increase in mRNA of the antiapoptotic Bcl-W and Bcl-XL, further suggesting the potential neuroprotective/antiapoptotic features of rasagiline. This is supported by the inventors recent results that have shown the involvement of Bcl-2 in neuroprotection by rasagiline, by the fact that rasagiline increased the expression of Bcl-2 in SH-SY5Y cells.

These results may be relevant with the fact that the Bcl2-related protein family regulate the mitochondrial membrane permeability transition (PT) pore triggers dissipation of mitochondrial membrane potential (ΔΨm), release of cytochrome C and apoptosis-inducing factor from the mitochondrial intermembrane space and other downstream events leading to cell death. Indeed, the inventors' previous data have implied that rasagiline suppressed cell death through direct interaction with mitochondrial apoptosis cascade and may contribute to the maintenance of the (ΔΨm) found with rasagiline treatment of neurons entering apoptosis.

### REFERENCES

Akao, Y., Nakagawa, Y., Maruyama, W., Takahashi, T., and Naoi, M. (1999) Apoptosis induced by an endogenous neurotoxin, N-methyl(R)salsolinol, is mediated by activation of caspase-3. Neurosci. Lett. 267:153-156.
Akao, Y., Maruyama, W., Shimizu, S., Yi, H., Nakagawa, Y., Shamoto-Nagai, M., Youdim, M. B. H., Tsujimoto, Y., and Naoi, M. (2002a) Mitochondrial permeability transition mediates apoptosis induced by N-methyl(R)salsolinol, an endogenous neurotoxin, and is inhibited by Bcl-2 and Rasagiline, N-Propargyl-1(R)-aminoindan. J. Neurochem. 82: 913-923.

Akao, Y., Maruyama, W., Yi, H., Shamoto-Nagai, M., Youdim, M. B. H., and Naoi, M. (2002b) An anti-Parkinson's disease drug, N-propargyl-1(R)-aminoindan (rasagiline), enhances expression of anti-apoptotic Bcl-2 in human dopaminergic SH-SY5Y cells. Neurosci. Lett. 326: 105-108.

Durden D A, Dyck L E, Davis B A, Liu Y D, Boulton A A. (2000) Metabolism and pharmacokinetics, in the rat, of (R)-N-(2-heptyl)methyl-propargylamine (R-2HMP), a new potent monoamine oxidase inhibitor and antiapoptotic agent. Drug Metab Dispos. 28(2):147-54.
Finberg, J P, Lamensdorf, I, Weinstock, M, Schwartz, M, and Youdim, M B H. (1999) Pharmacology of rasagiline (N-propargyl-1R-aminoindan). Adv. Neurol. 80: 495-499.
Grossberg, G., and Desai, A. (2001) Review of rivastigmine and its clinical applications in Alzheimer's disease and related disorders. Expert Opin. Pharmacother. 2: 653-666.
Huang, W, Chen, Y, Shohami, E, and Weinstock, M. (1999) Neuroprotective effect of rasagiline, a selective monoamine oxidase-B inhibitor, against closed head injury in the mouse. Eur. J. Pharmacol. 366: 127-135.
Maruyama, W, Akao, Y, Youdim, M B H, and Naoi, M. (2000a) Neurotoxins induce apoptosis in dopamine neurons: protection by N-propargylamine-1(R)-and (S)-aminoindan, rasagiline and TV1022. J. Neural Transm. Suppl, 171-186.
Maruyama, W, Youdim, M B H, and Naoi, M. (2000b) Antiapoptotic function of N-propargylamine-1(R)-and (S)-aminoindan, Rasagiline and TV1022. Ann N Y Acad Sci 939:320-329.
Maruyama, W., Boulton, A. A., Davis, B. A., Dostert, P., and Naoi, M. (2001a) Enantio-specific induction of apoptosis by an endogenous neurotoxin, N-methyl(R)salsolinol, in dopaminergic SH-SY5Y cells: suppression of apoptosis by N-(2-heptyl)-N-methylpropargylamine. J. Neural Transm. 108: 11-24.
Maruyama, W, Akao, Y, Youdim, M B H, Boulton, A A, Davis, B A, and Naoi, M. (2001b) Transfection-enforced Bcl-2 overexpression and an anti-Parkinson drug, rasagiline, prevent nuclear accumulation of glyceraldehyde-3 phosphate dehydrogenase induced by an endogenous dopaminergic neurotoxin, N-methyl(R)salsolinol. J. Neurochem. 78: 727-735.
Maruyama, W, Youdim, M B H, and Naoi, M. (2001c) Antiapoptotic properties of rasagiline, N-propargylamine-1(R)-aminoindan, and its optical (S)-isomer, TV1022. Ann N Y Acad Sci 939: 320-329.
Maruyama, W., Takahashi, T., Youdim, M. B. H., and Naoi, M. (2002) The anti-Parkinson drug, rasagiline, prevents apoptotic DNA damage induced by peroxynitrite in human dopaminergic neuroblastoma SH-SY5Y cells. J. Neural Transm. 109: 467-481.
Speiser, Z, Mayk, A, Eliash, S, and Cohen, S. (1999) Studies with rasagiline, a MAO-B inhibitor, in experimental focal ischemia in the rat. J. Neural Transm. 106: 593-606.
Tatton W G. (1993) Selegiline can mediate neuronal rescue rather than neuronal protection. Movement Disorders 8 (Supp. 1):S20-S30.
Tatton W G, Greenwood C E. (1991) Rescue of dying neurons: a new action for deprenyl in MPTP parkinsonism. J Neurosci Res. 30:666-672.

Tatton, W G, Chalmers-Redman, R M, Ju, W J, Mammen, M, Carlile, G W, Pong, A W, and Tatton, N A. (2002) Propargylamines induce antiapoptotic new protein synthesis in serum- and nerve growth factor (NGF)-withdrawn, NGF-differentiated PC-12 cells. J. Pharmacol. Exp. Ther. 301: 753-764.
Weinstock, M. (1999) Selectivity of cholinesterase inhibition: Clinical implications for the treatment of Alzheimer's disease. CNS Drugs 12: 307-323.
Yogev-Falach, M., Amit, T., Bar-Am, O., Sagi, Y., Weinstock, M., and Youdim, M B H. (2002) The involvement of mitogen-activated protein (MAP) kinase in the regulation of amyloid precursor protein processing by novel cholinesterase inhibitors derived from rasagiline. FASEB J. 16:1674-1676.
Youdim, M B H. (1991) PC12 cells as a window for the differentiation of neural crest into adrenergic nerve ending and adrenal medulla. J. Neural Transm. Suppl. 34: 61-67.
Youdim, M B H, and Weinstock, M. (2002a) Novel neuroprotective anti-Alzheimer drugs with antidepressant activity derived from the anti-Parkinson drug, rasagiline. Mechanisms of Ageing & Developments 123: 1081-1086.
Youdim, M B H, and Weinstock, M. (2002b) Molecular basis of neuroprotective activities of rasagiline and the anti-Alzheimer drug, TV3326. ((N-propargyl-(3R)aminoindan-5-yl)-ethyl methyl carbamate) Cell. Mol. Neurobiol. 21: 555-573.
Youdim, M B H, Gross, A, and Finberg, J P M. (2001a) Rasagiline [N-Propargyl-1R(+)-aminoindan], a selective and potent inhibitor of mitochondrial monoamine oxidase B. Br. J. Pharmacol. 132: 500-506.
Youdim, M B H, Wadia, A, Tatton, N A, and Weinstock, M. (2001b) The anti-Parkinson drug rasagiline and its cholinesterase inhibitor derivatives exert neuroprotection unrelated to MAO inhibition in cell culture and in vivo. Ann N Y Acad Sci 939: 450-458.
Youdim, M B H, Amit, T, Yogev-Falach, M, Bar-Am, O, Maruyama, W, and Naoi, M. (2003) The essentiallity of Bcl-2, PKC and proteasome-ubiquitin complex activations in the neuroprotecitve-antiapoptotic action of the anti-Parkinson drug, rasagiline. Biochem. Pharmacol. 66(8):1635-41.

1. A method for treating a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury which comprises administering to an individual in need an amount of propargylamine or a pharmaceutically acceptable salt thereof effective to treat said disease, disorder or condition in the individual.

2. A method according to claim 1, wherein said neurodegenerative disease is Parkinson's disease, Huntington's disease or amyotrophic lateral sclerosis.

3. A method according to claim 1, wherein said dementia is Alzheimer's disease or a non-Alzheimer's dementia selected from the group consisting of Lewy body dementia, vascular dementia and a dementia caused by Parkinson's disease, Huntington's disease, Creutzfeldt-Jakob disease, head trauma or HIV infection.

4. A method according to claim 1, wherein said affective or mood disorder is depression, a dysthymic disorder, a bipolar disorder, a cyclothymic disorder, schizophrenia or a schizophrenia-related disorder selected from the group consisting of brief psychotic disorder, a schizophreniform disorder, a schizoaffective disorder and delusional disorder.

5. A method according to claim 1, wherein said drug use and dependence is alcoholism, opiate dependence, cocaine dependence, amphetamine dependence, hallucinogen dependence, or phencyclidine use and withdrawal symptoms related thereto.

6. A method according to claim 1, wherein said memory loss disorder is amnesia or memory loss associated with Alzheimer's type dementia, with a non-Alzheimer's type dementia, or with a disease or disorder selected from the group consisting of Parkinson's disease, Huntington's disease, Creutzfeldt-Jakob disease, head trauma, HIV infection, hypothyroidism and vitamin B12 deficiency.

7. A method according to claim 1, wherein said acute neurological traumatic disorder or neurotrauma is head trauma injury or spinal cord trauma injury.

8. A method according to claim 1, wherein said demyelinating disease is multiple sclerosis.

9. A method according to claim 1, wherein said seizure disorder is epilepsy.

10. A method according to claim 1, wherein said cerebrovascular disorder is brain ischemia or stroke.

11. A method according to claim 1, wherein said behavior disorder is of neurological origin and is a hyperactive syndrome or an attention deficit disorder.

12. A method according to claim 1, wherein said neurotoxic injury is caused by a neurotoxin and said neurotoxin is a nerve gas or the toxin delivery system of poisonous snakes, fish or animals.

13. A method according to claim 10 for treatment of brain ischemia or stroke which comprises administering to an individual in need an amount of propargylamine or a pharmaceutically acceptable salt thereof effective to treat brain ischemia or stroke in said individual.

14. A method according to claim 7 for treatment of head trauma injury which comprises administering to an individual in need an amount of propargylamine or a pharmaceutically acceptable salt thereof effective to treat head trauma injury in said individual.

15. A method according to claim 7 for treatment of spinal cord trauma injury in an individual which comprises administering an amount of propargylamine or a pharmaceutically acceptable salt thereof effective to treat spinal cord trauma injury in the individual.

16. A method according to claim 7 for treatment of neurotrauma in an individual which comprises administering to the individual an amount of propargylamine or a pharmaceutically acceptable salt thereof effective to treat neurotrauma in the individual.

17. A method according to claim 2 for treatment of an individual afflicted with a neurodegenerative disease which comprises administering to the individual an amount of propargylamine or a pharmaceutically acceptable salt thereof effective to treat the neurodegenerative disease in the individual.

18. A method according to claim 17 wherein said neurodegenerative disease is Parkinson's disease.

19. A method according to claim 3 for treatment of an individual afflicted with a dementia, which comprises administering to the individual an amount of propargylamine or a pharmaceutically acceptable salt thereof effective to treat the dementia in the individual.

20. A method according to claim 19 wherein said dementia is Alzheimer's disease.

21. A method according to claim 12 for treatment of an individual afflicted with a neurotoxic injury which comprises administering to the individual an amount of propargylamine or a pharmaceutically acceptable salt thereof effective to treat the neurotoxic injury in the individual.

22. A method according to claim 21 wherein said neurotoxic injury is caused by nerve gases.

23. A method according to claim 4 for treatment of an individual afflicted with depression, which comprises administering to the individual an amount of propargylamine or a pharmaceutically acceptable salt thereof effective to treat depression in the individual.

24. An article of manufacture comprising packaging material and a pharmaceutical composition contained within the packaging material, said pharmaceutical composition comprising propargylamine or a pharmaceutically acceptable salt thereof, and said packaging material includes a label that indicates that said agent is therapeutically effective for treating a disease, disorder or condition selected from the group consisting of: (i) a neurodegenerative disease, (ii) a dementia; (iii) an affective or mood disorder; (iv) drug use and dependence; (v) a memory loss disorder; (vi) an acute neurological traumatic disorder or neurotrauma; (vii) a demyelinating disease; (viii) a seizure disorder; (ix) a cerebrovascular disorder; (x) a behavior disorder; and (xi) a neurotoxic injury.


SEQUENCE LISTING (20 sequences)

SEQ ID NO: 1 - DNA, 20 bp, ARTIFICIAL SEQUENCE
  SYNTHETIC
gcttagcccttttcgaggac

SEQ ID NO: 2 - DNA, 19 bp, ARTIFICIAL SEQUENCE
  SYNTHETIC
gatcccaccaggactggat

SEQ ID NO: 3 - DNA, 20 bp, ARTIFICIAL SEQUENCE
  Synthetic
tgcagaggatgattgctgac

SEQ ID NO: 4 - DNA, 20 bp, ARTIFICIAL SEQUENCE
  SYNTHETIC
ggaggaagtccagtgtccag

SEQ ID NO: 5 - DNA, 19 bp, ARTIFICIAL SEQUENCE
  SYNTHETIC
gctgaggcagaagggttat

SEQ ID NO: 6 - DNA, 20 bp, ARTIFICIAL SEQUENCE
  SYNTHETIC
tggaaaagttcgtcggaaac

SEQ ID NO: 7 - DNA, 20 bp, Artificial Sequence
  synthetic
ggtgagtcggattgcaagtt

SEQ ID NO: 8 - DNA, 20 bp, Artificial Sequence
  SYNTHETIC
gagcccagcagaactacacc

SEQ ID NO: 9 - DNA, 19 bp, Artificial Sequence
  SYNTHETIC
gactctggagagcgtgaat

SEQ ID NO: 10 - DNA, 20 bp, Artificial Sequence
  SYNTHETIC
ccactcgctaatactgtcac

SEQ ID NO: 11 - DNA, 24 bp, Artificial Sequence
  SYNTHETIC
tgaaccctcagtggaatgagtcct

SEQ ID NO: 12 - DNA, 24 bp, Artificial Sequence
  SYNTHETIC
atggctgcttcctgtcttctgaag

SEQ ID NO: 13 - DNA, 20 bp, Artificial Sequence
  SYNTHETIC
ttcttcaagcagccaacctt

SEQ ID NO: 14 - DNA, 20 bp, Artificial Sequence
  SYNTHETIC
tgtagctgtgcagacggaac

SEQ ID NO: 15 - DNA, 20 bp, Artificial Sequence
  SYNTHETIC
aaatagagagcgcagcctga

SEQ ID NO: 16 - DNA, 20 bp, Artificial Sequence
  SYNTHETIC
taaacaaggccgaatgttcc

SEQ ID NO: 17 - DNA, 20 bp, Artificial Sequence
  SYNTHETIC
ccccttgtgaccaggaacta

SEQ ID NO: 18 - DNA, 20 bp, Artificial Sequence
  SYNTHETIC
gcctttgcctaacaccttga

SEQ ID NO: 19 - DNA, 20 bp, Artificial Sequence
  SYNTHETIC
gtaacccgttgaaccccatt

SEQ ID NO: 20 - DNA, 20 bp, Artificial Sequence
  SYNTHETIC
ccatccaatcggtagtagcg